(19) United States
(12) Patent Application Publication
(10) Pub. No.: US 2018/0000408 A1
(43) Pub. Date: Jan. 4, 2018

(54) BABY SLEEP MONITOR

(71) Applicant: KONINKLIJKE PHILIPS N.V., EINDHOVEN (NL)

(72) Inventors: ADRIENNE HEINRICH, EINDHOVEN (NL); PEDRO MIGUEL FONSECA, EINDHOVEN (NL)

(21) Appl. No.: 15/533,388

(22) PCT Filed: Dec. 8, 2015
(86) PCT No.: PCT/EP2015/078900

§ 371 Date: Jun. 6, 2017

(30) Foreign Application Priority Data
Dec. 16, 2014 (EP) 14198246.2

(51) Int. Cl. A61B 5/00; A61B 5/0205; A61B 5/11; A61B 5/08; A61B 5/024

(52) U.S. Cl. CPC A61B 5/4809; A61B 5/4812; A61B 5/7267; A61B 5/0205; A61B 5/024; A61B 5/08; A61B 2503/04; A61B 5/1103

(57) ABSTRACT

A sleep monitor for monitoring baby sleep uses sleep state classification based on heartbeat feature respiration features. The sleep monitor automatically retrains the classification during use of the sleep monitor. Training examples for use in this training process are generated automatically by detecting time instants whereat the baby in the bed is in a wake state, based on signals from the at least one of a sound feature detector a movement feature detector (112) and an open eye detector (114). The retraining may comprise using time sequence from the end of detection of wake states to assign a class to heartbeat feature and/or respiration feature values during that time sequence for the training process. In an embodiment, the retraining comprises clustering detected heartbeat feature and/or respiration feature values detected outside the detected wake states.

### FIELD OF THE INVENTION

The invention relates to a baby sleep monitor and to a method of monitoring a sleeping baby.

### BACKGROUND OF THE INVENTION

WO2005055802 discloses a sleep guidance system designed to monitor a person's sleep stage and to guide the person to selected sleep stages. The sleep stages of normal adult human sleep include stages such as a one or more “deep sleep” stages, a “rapid eye movement” sleep stage etc. Conventionally, the sleep stage is determined based on electroencephalograph (EEG) measurements. However, other physiological measurements on a person can also be used to distinguish different sleep stages. WO2005055802 mentions electrooculograms, electromyograms, electroencephalographs and other polysomnography monitors, microphones, motion sensors, moisture sensors, muscle tension monitors, blood pressure cuffs, respirators, pulse oximeters, thermometers, and the like and gives examples of 2 heart rate, respiration and temperature changes between sleep stages.

WO2005055802 discloses that prior calibration of a personalized sleep profile may provide better monitoring results. Calibration of the relation between particular sleep patterns and physiological characteristics of a sleeper can be used to establish the personalized sleeper profile. The personalized sleeper profile may be stored in association with a processor. The processor uses the personalized sleeper profile to control how physiological characteristics are used to determine the sleep state and optionally whether the sleeper is about to transition to a particular sleep stage.

For the calibration the processor monitors the sleep patterns and/or physiological characteristics of a sleeper. The processor of WO2005055802 evaluates which patterns of physiological characteristics occur at which portions of the sleeper's sleep cycle or under which circumstances and which physiological characteristics most clearly indicate a change between the sleeper's sleep stages. WO2005055802 also discloses that the responses of the sleeper to application of different stimuli can be calibrated, e.g. for use in sleep guidance.

Human baby sleep is very different from human adult sleep. Only two baby sleep states are distinguished: “active sleep” and “quiet sleep” and of course babies are also often in various “awake” states. A newborn sleeps in sleep cycles in which the active sleep state and quiet sleep state alternate. When a newborn first falls asleep, it enters immediately into “active sleep”. This is a relatively restless sleep state similar to REM (rapid eye movement) sleep in adults. Just as adults are more likely to awaken during REM, newborns are more likely to awaken during active sleep. Newborns may remain in this active sleep state for 25 minutes or more, after which they slip into a deeper sleep state known as “quiet sleep”. Compared to active sleep, quiet sleep is characterized by slower, more rhythmic breathing, little movement, and no eyelid fluttering. After about 50 minutes, a new sleep cycle with active sleep followed by quiet sleep occurs. Babies are less likely to awaken during quiet sleep than during active sleep.

The inventors have found that prior calibration of the relation between heartbeat features and/or respiration features and optionally other detected features on one hand and the “active sleep” and “quiet sleep” states on the other hand can be used to detect these sleep states. An advantage of heartbeat features and respiration features is that they can be detected by remote sensing without encumbering the baby. Optionally, detected baby movement features can be used as well, although this does not relieve the need for calibration. Baby movement features can also be detected without encumbering the baby. In any case, calibration remains necessary if such features are used for sleep state detection. Unfortunately, it was found that such calibration results provide reliable results only for a limited time after calibration. After that sleep classification results become unreliable. The inventors surmise that this is because development of the baby significantly affects the relation between the heartbeat features and the respiration features and the sleep states. These changes don't appear to be predictable based on the baby's age. This may be because different babies develop at different speed.

Frequently repeated recalibration of this relation has been found to make the detection of sleep stages in babies more reliable. However, recalibration is cumbersome if it involves more intrusive measurements like electroencephalograph (EEG) measurements or input of human determinations of sleep stages in order to compile the recalibrated relation.

### SUMMARY OF THE INVENTION

Among others, it is an object to provide for a sleep monitor that is capable of monitoring baby sleep in a period in which the baby develops, without requiring cumbersome recalibration.

A sleep monitor for monitoring baby sleep is provided that comprises
a heartbeat feature detector and/or a respiration feature detector;
a heartbeat feature and/or respiration feature based sleep state classifier with an input coupled to the heartbeat feature detector and/or the respiration feature detector;
at least one of a sound feature detector, a movement feature detector and an open eye detector;
a processing circuit configured to repeatedly execute a retraining process of the sleep state classifier during use of the sleep monitor, wherein the processing circuit is configured to detect time instants whereat the baby in the bed is in a wake state based on signals from the at least one of a sound feature detector, a movement feature detector and an open eye detector, and to use the detected time instants to generate or select training examples for the retraining process.

The sound feature detector may comprise a microphone located to pick-up sound originating in the baby bed. The movement feature detector may comprise a camera coupled to a video motion detector, an accelerometer, a radar and/or a force sensor. The open eye detector may comprise a camera coupled to a face detector. The heartbeat feature detector and respiration feature detector may comprise a camera, a Doppler radar, a force sensor, and/or an accelerometer etc.

A conventional feature based classifier from the field of pattern recognition may be used, as well as a conventional classifier training process from that field. In the sleep monitor for monitoring baby sleep, the training process is applied repeatedly during use, that is, following classification based on earlier training results. It has been discovered that in the case of baby sleep monitoring retraining is necessary to obtain long term reliable results and by doing so automatically during use no cumbersome adjustments are needed. Although the classification is based on heartbeat and/or respiration features and possibly additional features such as baby movement features, the reliability of the training process is improved by using other detectable effects like sound due to crying, large motion and/or detection that the eyes of the baby are open. Direct observation of such effects makes it possible to provide more reliable detection of time instants when the baby is awake. By using this information in the selection or generation of training examples for use in the retraining process the retraining is made more reliable. The retraining may comprise using time sequence from the end of detection of wake states to assign a class to heartbeat feature and/or respiration feature values during that time sequence for the training process. The retraining may also comprise clustering detected heartbeat and/or feature respiration feature values detected outside the detected wake states.

In an embodiment, the processing circuit is configured to detect the non-sleep state based on whether a movement amplitude of a baby motion feature detected by the movement feature detector exceeds a first predetermined value, a loudness property of sound detected by the sound feature detector exceeds a second predetermined value, and/or the open eye detector detects an open eye on the baby in the bed. Large movements, especially of the torso, as observed e.g. by matching image content in mutually displaced image areas in successively captured images and determining the offset, or from accelerometer, force sensor or radar measurements, can be used to increase the reliability of detection of a wake state. Large detected movements may also indicate that a parent puts the baby in bed, which indicates a high likelihood that the baby is in a wake state. Loud sounds that can be attributes to crying from the baby are a reliable indicator of a wake state. Similarly, detection that the baby has its eyes open, by detecting a face in an image of the baby and detecting visibility of the irises of the eye in the face is a reliable indicator of a wake state.

For the evaluation of sleep, the main purpose of a baby sleep monitor is to distinguish between a plurality of different sleeping states, i.e. different states while the baby is asleep or optionally awake, while the baby is in bed (as used herein a sleep state may be used to indicate whether the baby is sleeping or not, and in the former case in which of the active and quiet sleeping states it is sleeping). Preferably, retraining comprises retraining the criteria for distinguishing between the different sleeping states.

In an embodiment, the processing circuit is configured to exclude a training example for use to train classification criteria for distinguishing between said plurality of sleeping states and the awake state, based on whether a measurement time interval used to obtain the training example comprises at least one of the detected time instants. By eliminating such training examples, a subset of training examples is obtained that contains a higher fraction of examples with heartbeat and/or respiration features from sleeping states, if not only examples from sleeping states. Use of such a subset for training makes it possible to realize a more reliable distinction between different sleeping states.

In an embodiment, the processing circuit being configured to provide training examples associated with the quiet sleep obtained for training time intervals that follow directly after the detected time instants whereat the baby is in a non-sleep state. For the retraining process at least part of the examples may be provided in association with the state to which the training process should be classified. Because it is known that a baby is most likely to enter the active sleep state after being awake, the detection of the time instants when the baby was awake can be used to provide associations of training examples with that sleeping state.

A method of automatically monitoring baby sleep is provided with the steps of
detecting heartbeat features, movement features and/or respiration features of a baby for successive measurement time intervals;
automatically classifying sleep states of the baby associated with the successive measurement time intervals based on the heartbeat and/or respiration features of the measurement time intervals;
automatically repeatedly retraining the classification criteria used for said classifying during use, said retraining comprising
detecting time instants whereat the baby in the bed is in a wake state based on signals from at least one of a sound feature detector, a movement feature detector and an open eye detector,
using the detected time instants to generate or select training examples for the retraining.

In each embodiment classification may be based on an implicit or explicit definition of ranges of the heartbeat feature values, or ranges of respiration feature values, or ranges of combinations of heartbeat feature and respiration feature values, or optionally ranges of any of these combined with values of other features. Similarly, classification may be based on an implicit or explicit definition of a function or functions of such values or combination of values, the function or functions expressing the likelihood of different states. Heartbeat feature and/or respiration feature based classification may come down to a determination of the defined range, or the most likely range, in which the heartbeat and/or respiration feature value from the measurement time interval is located.

In such embodiments, retraining may comprise adjusting parameters that define the ranges or the function or functions. Parameters representing central values of ranges and/or boundaries of the ranges may be adjusted for example. In another example, the function or functions may depend on distances to adjustable reference values like the central values.

In other embodiments the classification assigned to a measurement time interval may also depend on the feature values from surrounding time intervals. For example, the classifications may be based on the most likely state in a time dependent model, such as a hidden Markov model, that takes account of the likelihood of transitions between different states and relates states to the likelihood of observed feature values. The trained functions for the likelihoods of different states may be used in such models to find the states, and/or the likelihoods of transitions may be adjusted based on sequences of training examples.

A computer program product, such as a computer readable medium, is provided that comprises machine readable instructions for a programmable data processing system that, when executed by the data processing system, will cause the data processing system to execute the method.

### BRIEF DESCRIPTION OF THE DRAWINGS

These and other objects and advantageous aspects will become apparent from a description of exemplary embodiments with reference to the accompanying figures.

FIG. 1 shows a baby sleep monitor.

FIG. 1a shows a modular diagram of a baby sleep monitor

FIG. 2 shows a flow chart of baby sleep monitoring

FIG. 3 shows an example of a state diagram of a model.

FIG. 4 shows a flow chart of an exemplary embodiment of a training process.

FIG. 5 shows a flow chart of an exemplary embodiment of a training process.

### DETAILED DESCRIPTION OF EMBODIMENTS

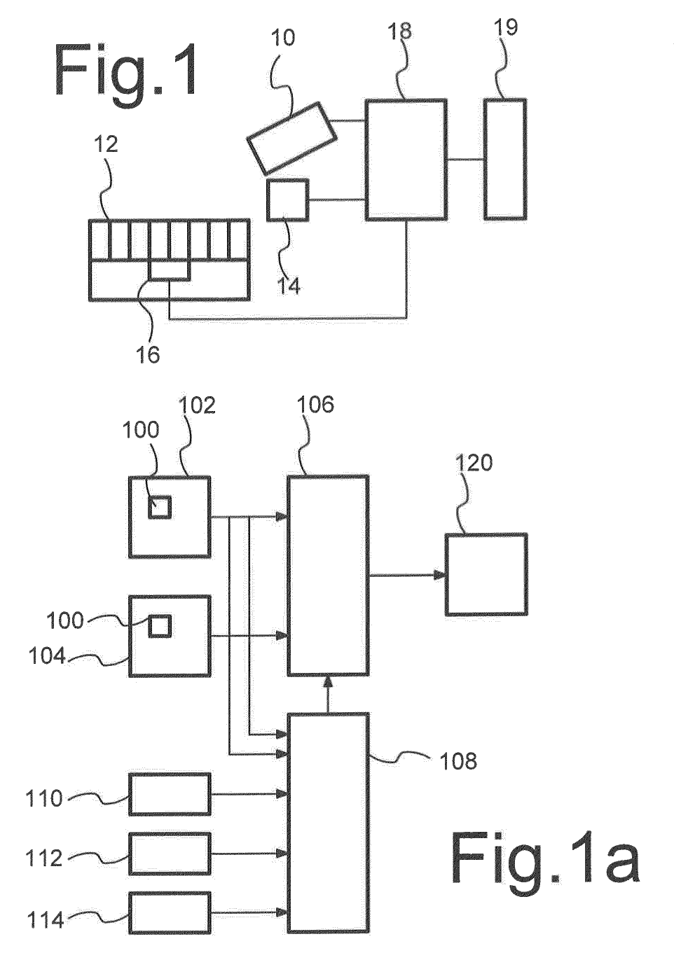

FIG. 1 shows an exemplary baby sleep monitor. The baby sleep monitor comprises a camera 10 directed at a bed 12, a microphone 14, a force sensor 16, a data processing system 18 and a display 19. Force sensor 16 is coupled to bed 12, and arranged to measure a force as a function of time due to the weight of a baby on the bed and weight force or pressure changes accelerations associated with its movements. Camera 10, microphone 14, force sensor 16 and display 19 are coupled to data processing system 18.

In operation, the baby sleep monitor is used to determine baby sleep states as a function of time and accumulate statistics of these sleep states.

When the necessary equipment is available, different sleep states can be distinguished directly based on electro encephalogram measurements and a number of similar measuring techniques. For baby sleep usually only two different sleep states are used, labeled quiet sleep and active sleep. However, the measurement set-up for such direct measurements is cumbersome and therefore unsuitable for daily use or use by nonprofessionals such as most parents.

Instead the present baby sleep monitor uses movement, heartbeat and respiration feature values to estimate which baby sleep states apply. Heartbeat and respiration feature values can be detected in a less cumbersome way, for example by remote camera sensing, weight force, acceleration or Doppler measurements. In the case of babies there is no unique general relation between such feature values and the sleep states that would result from using electro encephalogram measurements. Instead data processing system 18 determines the relation adaptively by means of a training process. Data processing system 18 determines updated definition of these ranges or functions repeatedly by a training process performed by data processing system 18 in order to track changes in the relation due to development of the baby.

FIG. 1a shows a modular diagram of the processing system of the baby sleep monitor, comprising a heartbeat feature detector 102, a respiration feature detector 104, a classifier 106, a training module 108, a sound feature detector 110, a movement feature detector 112, an open eye detector 114 and a data analysis module 120. Heartbeat feature detector 102 and respiration feature detector 104 have outputs coupled to classifier 106. Classifier 106 has an output coupled to data analysis module 120. Heartbeat feature detector 102, respiration feature detector 104, sound feature detector 110, movement feature detector 112 and open eye detector 114 have outputs coupled to training module 108. Training module 108 has an output coupled to classifier 106.

Heartbeat feature detector 102, respiration feature detector 104, sound feature detector 110, movement feature detector 112 and open eye detector 114 comprise sensors 100 (shown only in heartbeat feature detector 102 and respiration feature detector 104), or be coupled to sensors. Furthermore, they comprise circuits to process data from those sensors. Alternatively, they may be realized using software modules executed by data processing system 18. The circuits for processing data may be realized using a programmable data processor in combination with software modules. In this implementation, FIG. 1a may be seen as a schematic software architecture. Similarly, classifier 106, training module 108 and data analysis module 120 may be realized by means of the data processor and software modules. Although an embodiment with all of heartbeat feature detector 102, respiration feature detector 104, sound feature detector 110, movement feature detector 112 and open eye detector 114 are shown by way of example, it should be realized that in other embodiments only subsets of these detectors may be present.

In operation, heartbeat feature detector 102 uses sensor data to measure one or more heartbeat features, such as heart beat frequency, heart beat cycle duration, heart beat frequency histograms, heart rate variability etc. in successive measurement time intervals. Respiration feature detector 104 uses sensor data to measure one or more respiration features, such as respiration frequency, respiration cycle duration, respiration frequency histograms, respiration variability etc. in the successive measurement time intervals. Classifier 106 selects sleep states based on at least one of the heartbeat and respiration features. Classifier 106 signals the selected sleep states to data analysis module 120, which collects statistics of the sleep states and/or generates alerts based on the selected sleep states.

Training module 108 repeatedly executes a retraining process of the classifier 106 during use of the sleep monitor. Training module 108 detects time instants whereat the baby in the bed is in a wake state based on signals from the at least one of sound feature detector 110, movement feature detector 112 and open eye detector 114. Training module 108 uses the detected time instants to generate or select training examples for the retraining process. Training module 108 then uses the training examples to select parameters that define classification by classifier 106, and loads these parameters into classifier 106.

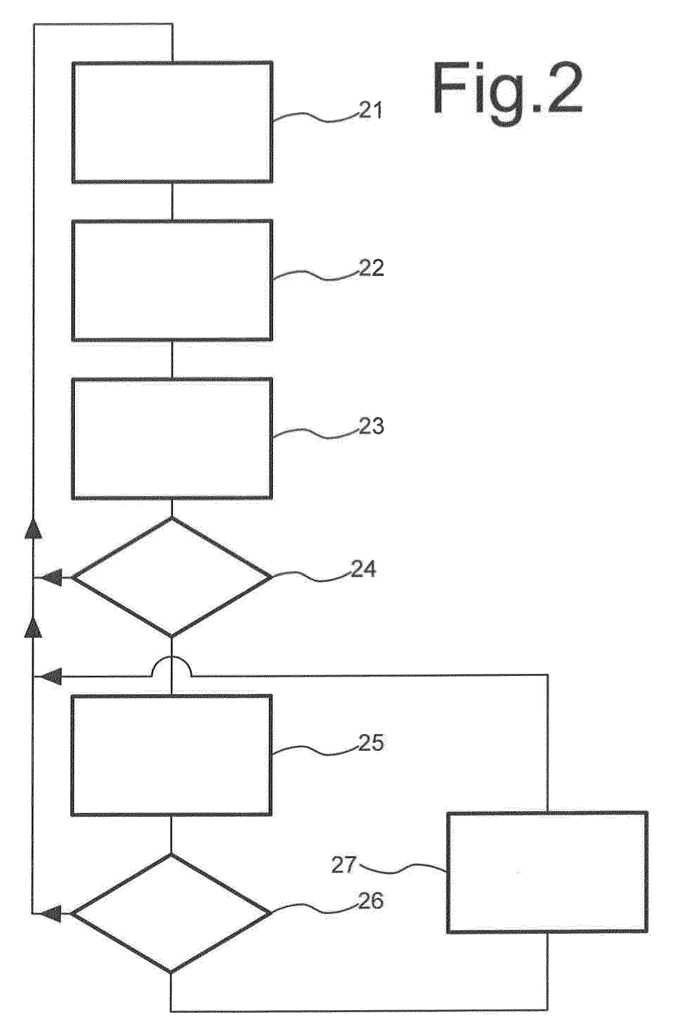

FIG. 2 shows a flow chart of baby sleep monitoring by means of heartbeat and respiration feature values. In a first step 21 data processing system 18 (heartbeat feature detector 102 and respiration feature detector 104) measures heartbeat and respiration features and optionally movement features in a measurement time interval. In an embodiment, data processing system 18 used image data obtained from camera 10 for this purpose.

Heartbeat feature detector 102 may measure heartbeat e.g. from the effect of periodic forces exerted on the bed due to heartbeat, with a period duration in a range corresponding to heartbeat, as detected by a force or acceleration sensor coupled to the bed. It can also be measured from periodic movement, as detected by Doppler radar, or its effect on the variation of intensity of light reflection by the skin, e.g. reflected color or grey level intensity. The degree of blood perfusion of the skin varies during the hearth beat cycle. Accordingly, data processing system 18 may be configured to collect pixel values (r averages of pixel values) in an area of the images from camera 10 that shows skin of the baby in bed 12. In alternative examples, or in addition a Doppler radar, LIDAR, a force (weight) sensor or an accelerometer may be used to measure movements, forces or accelerations due to heart beat. A force sensor or accelerometer may be placed on or under the mattress, e.g. at a location in a vicinity of where the chest of the baby will be located. In other embodiments clip-on sensors for use on the baby may be used. A force sensor or accelerometer may use that are oriented to respond to force changes or accelerations in a vertical direction, i.e. perpendicular to the plane on which the baby lies.

From the results obtained for a temporal series of images, Doppler radar, LIDAR, force and/or acceleration sensing results and data processing system 18 may determine the duration of time between corresponding features of the pixel variation as a function of time, and/or a frequency. The resulting measurements of color, speed, force or acceleration as a function of time may be temporally filtered to emphasize the periodic effect of heartbeat. The duration between successive minima or maxima in the pixel values may be determined for example, by detecting the time points of minima or maxima and determining the difference. Similarly the duration between minima or maxima or zero crossings of the measured speed, force or acceleration may be measured. The duration and/or frequency may be used as heartbeat feature, or data processing system 18 may derive one or more heartbeat features from a plurality of successively measured durations of frequencies, e.g. by averaging the duration and/or computing spread in the duration such as its variance, heart rate variability or the size of the range of variation of the duration between heart beats. The average or spread in a measurement time interval of between one and ten minutes may be determined for example. As another possibility, a Fourier transform of the pixel values may be taken over a measurement time interval and the spectral distribution over predetermined spectral bands in the Fourier transform may be used as a heartbeat feature.

Respiration feature detector 104 may measure effects of respiration can be measured from movement observed in the images, or in radar or lidar signals for example. Respiration leads to periodic chest movements that result in periodic displacements of image features that are visible in the camera images or movement observed by radar etc, in areas of those images where the chest or clothes on the chest are visible. Accordingly, data processing system 18 may use the output of a conventional motion vector detector or to compare image data in areas of successive images to determine displacement of corresponding image features between the successive images. Correlation between images for successive time points as a function of distance in the images may be used for example. Data processing system 18 may apply a temporal filter to emphasize periodic effects of respiration in an expected range of frequencies of respiration. Data processing system 18 may determine the duration of time between corresponding features of the motion or frequency as a function of time. The duration between successive minima or maxima in the motion or its time derivative may be determined for example, by detecting the time points of minima or maxima and determining the difference, or from radar Doppler, force or acceleration measurements for example. This duration or frequency may be used as respiration feature, or data processing system 18 may derive one or more respiration features from a plurality of successively measured durations, e.g. by averaging the duration and/or computing its spread. As another possibility, a Fourier transform of the motion may be taken over a measurement time interval and the spectral distribution over predetermined spectral bands in the Fourier transform may be used as a respiration feature.

Optionally, data processing system 18 may determine further features from the images from camera 10, such as relative body part motions like finger motion relative to the hand, arm movement relative to the torso, leg movement relative to the torso etc. Data processing system 18 may detect motion vectors of body parts by searching for image areas with matching content in images captured at successive time point and determining the offset between the locations of these image areas. Data processing system 18 may determine associations between image areas and body parts based on the relative location of the image areas with respect to further image areas that are known to be associated with other body parts, such as the head, which may be located by face detection or the torso, which may be identified from the fact that it is the largest body part.

Optionally, data processing system 18 may determine features of signals from other sensors, such as from force sensor 16. By way of example a standard deviation of force value variations may be determined, or power densities in predetermined bands of a spectrum of force value variations.

In a second step 22 data processing system 18 (classifier 106) assigns an estimated sleep state and/or probabilities to feature vectors that each contain measured vector of heartbeat and respiration feature values in the measurement time interval and optionally other feature values in the measurement time interval, such as motion vectors associated with body parts. Basically, assignment of an estimated sleep state makes use of an explicit or implicit predetermined definition of ranges in the space of feature vectors of heartbeat and respiration feature values an optionally the other feature values, and state indications associated with these ranges. As more than one feature may be involved, the ranges may be multidimensional ranges, such as half-spaces, polygons, circles, spheres etc. In one example, the half-spaces and polygons may be defined implicitly by thresholds for weighted sums of feature values.

As will be explained, the explicit or implicit predetermined definition of ranges and their associated state indications are determined by a training process, which however is not needed for understanding the assignment process of FIG. 2.

Assignment of an estimated sleep state may comprise a determination in which explicitly or implicitly defined range the measured feature vector is located, and assigns the state that is associated with that range as the sleep state to the measurement time interval. The determination of the range in which the measured feature vector is located may be made for example based on an explicit definition of the range or by computing the function value(s) of one or more predefined characteristic functions applied to the measured feature vector containing the feature values, and comparing the result to a threshold value. In this case the characteristic functions are used to characterize the ranges implicitly. Similarly, the probabilities of estimated sleep states may be computed by computing predefined probability functions of the measured vector of feature values.

As will be explained, the definition of the characteristic functions and/or the probability functions may be determined by a training process. In further embodiments assignment of the estimated sleep state and/or probabilities may make use of measurements for a plurality of measurement time intervals. A hidden Markov model may be used for example, wherein the sleep states are states of the model and the vectors of heartbeat and respiration features are used as symbols that that result from these states with predetermined probabilities.

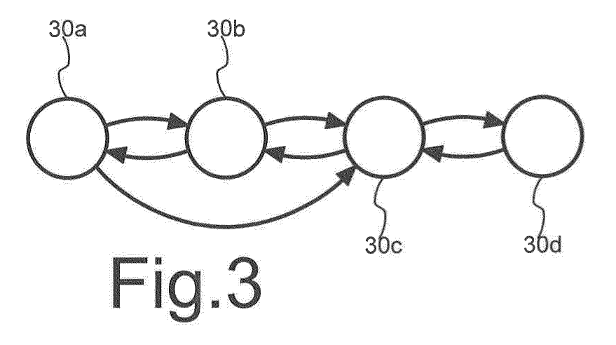

FIG. 3 shows an example of a state diagram of such a model. The state diagram represents states as nodes 30a-d, wherein a first node 30a represents a “baby not in bed” state. A second node 30b represents an “awake” state with the baby in bed 12. A third node 30c represents an “active sleep” state with the baby in bed. A fourth node 30d represents a “quiet sleep” state with the baby in bed 12. Optionally, a “no detection possible” state may be added, which occurs for example when parents obscure the image of the baby, or cause large forces on the bed. Solid arrows represent the transitions that most frequently result in the different states 30a-d. When the baby is put to bed, the awake or active sleep states are mostly reached. From the awake state, transitions mainly occur to the active sleep state. Transitions to the quiet sleep state mostly occur from the active sleep state and vice versa. Babies awake from the active or quite sleep state. Parents mostly take the baby from the bed in the awake state, when it is crying. In addition to the transitions indicated by the solid arrows other, less frequent, transitions (not shown) may be possible, such as that the baby enters the awake state directly from the quiet sleep state, or is put to be or taken out of bed when in one of the sleep states.

A hidden Markov model includes probability values for at least part of the transitions between the states and the probabilities of different symbols (e.g. measured heartbeat and breathing feature values) when in each state. Assignment based on a hidden Markov model comprises an inverse computation of the likelihood of being in the different states of the model based on the measured symbols and their time sequence. In this process provisional assignments of estimated sleep state and/or probabilities based on individual vectors of heartbeat and respiration features may be used as input for the assignment based on the time sequence of measured the symbols. As will be explained, the definition of parameters of the hidden Markov model may be determined by a training process.

In a third step 23 data processing system 18 (data analysis module 120) records the assigned estimated sleep state and/or probabilities in association with the measurement time interval, by causing them to be stored in a storage device that is part of data processing system 18, or located elsewhere. Optionally, data processing system 18 may store the underlying the measured vector of feature values. In this case second step 22 could be moved to a later stage.

In a fourth step 24 data processing system 18 tests whether display or an aggregation of sleep state assignments is needed, for example in response to input of a user instruction to display aggregated sleep data, and optionally whether a condition for generating an alarm signal is met. If not, data processing system 18 repeats the process from first step 21. Otherwise data processing system 18 proceeds to a fifth step 25.

In fifth step 25, data processing system 18 retrieves the recorded sleep state assignments over a selected time period, such as a selected number of hours from a current time, a night or a day. Data processing system 18 may be configured to cause display 19 to display assigned sleep states for measurement time intervals along a time scale. Although fifth step 25 is shown as a sequential step in the process, it may in fact be executed concurrently with the other steps, e.g. in a separate processing thread or by a different processor.

Data processing system 18 may be configured to aggregate sleep states in fifth step 25, e.g. by computing amounts of time spent in respective ones of the sleep states in the selected time period, e.g. based on counts of measurement time intervals assigned to the different sleep states, and/or by computing the lengths of continuous time intervals that span a plurality of measurement time intervals wherein a same sleep state was continuously assigned. Data processing system 18 may be configured to cause display 19 to display the computed aggregates, e.g. as numbers, bars or in the form of a histogram of the lengths of the continuous time intervals.

After fifth step 25, data processing system 18 executes a sixth step 26 wherein it determines whether or not to start a retraining process 27 for retraining the explicit or implicit ranges used for assignment in second step 22. Retraining (by training module 108) may be started periodically for example, or in response to detection of an indication of decreased reliability by classifier 106. Retraining may be executed concurrently with the process of FIG. 2: the old method of assignment may continue to be used in second step 22 until retraining is complete. Although sixth step 26 is shown as a sequential step in the process, it may in fact also be executed concurrently with the other steps, e.g. in a separate processing thread or by a different processor.

As described, the assignment of sleep state and/or probabilities of sleep states by data processing system 18 in second step 22 involves use of predetermined definitions of ranges of values of the vectors of heartbeat and respiration feature values and/or definitions of functions of those vectors and/or models that express the likelihood of sequences of vectors that used for the assignment.

It has been found that it is impossible to obtain reliable sleep state data using heartbeat and respiration feature values with fixed definitions. The relation between the sleep state as it can be determined by more direct methods and these feature values changes in the course of development of a baby and the timescale at which the changes occur and the way in which they occur vary widely between different babies.

To maintain a reliable sleep state assignment based on heartbeat and respiration feature values, data processing system 18 repeatedly performs a training process to determine updated definitions in the course of time. Training processes for determining definitions of ranges with associated state values, functions used to define such ranges implicitly, functions to assign probabilities and models such as hidden Markov models are known per se from the general field of pattern recognition.

In order to improve the reliability of the assignment of sleep states it would be preferable to use a supervised training process, that is, a training process wherein examples of measured vectors of feature values are provided, each in association with an indication of one of the state that pertained when the measured feature value was measured, or probabilities of different states.

However, supervised training is generally more cumbersome. Because it has been found that no single definition can be used for all babies, each repeated training process for baby sleep monitoring must be performed for an individual baby. It is not practicable to do so by applying electrodes to the baby in order to provide true state measurement based on electroencephalograms in combination with training examples of heartbeat and respiration features. Nor is it practicable to require parents to observe the baby for many hours and enter observed sleep states, after either learning to distinguish different baby sleep states.

Measurable context information can be used instead to support a form of supervised training that does not require application of electrodes to the baby or continued observation. Data processing system 18 may use input from microphone 14 to detect when the baby is crying. Detection of crying indicates that the baby is not in any of the sleep states. Similarly, data processing system 18 may use video input from camera 10 and/or a force sensor that measures force variations of forces exerted on the bed to detect when the baby performs large scale movements. Instead of camera images and/or sensed forces, radar, lidar or sonar measurements such as Doppler shift, transmission-reflection delay may be used, and/or accelerometer measurements. Like detection of crying, detection of movement above a sufficiently high threshold indicates that the baby is not in any of the sleep states. When such training examples are excluded from training the detection of sleep states, this type of context information increases the fraction of remaining training examples that correspond to actual sleep states, thereby increasing the reliability of the detection. Moreover, such training examples provide a form of supervised training information that the training examples are associated with the waking state.

In an embodiment, data processing system 18 may use such detection in a training process to eliminate exemplary heartbeat and respiration feature values from the training process that have been measured when the baby was detected not to be in a sleep state. This can be used to improve the accuracy of unsupervised training using the remaining exemplary heartbeat and respiration feature values. For example the remaining exemplary heartbeat and respiration feature values may be clustered into clusters that correspond more accurately to different sleep states because less noise from non-sleep states is present. In another example, the remaining exemplary heartbeat and respiration feature vectors may first be filtered to remove vectors that lie within clusters of the feature vectors that have been measured when the baby was detected not to be in a sleep state. Thus, more feature vectors that correspond to the awake state can be eliminated. In this embodiment the feature vectors that remain after filtering provide for more accurate training of the distinction between sleep states.

It should be emphasized that this embodiment is merely one example of a training process that makes use of context information. By way of example, flow chart of a training process will be given for this example.

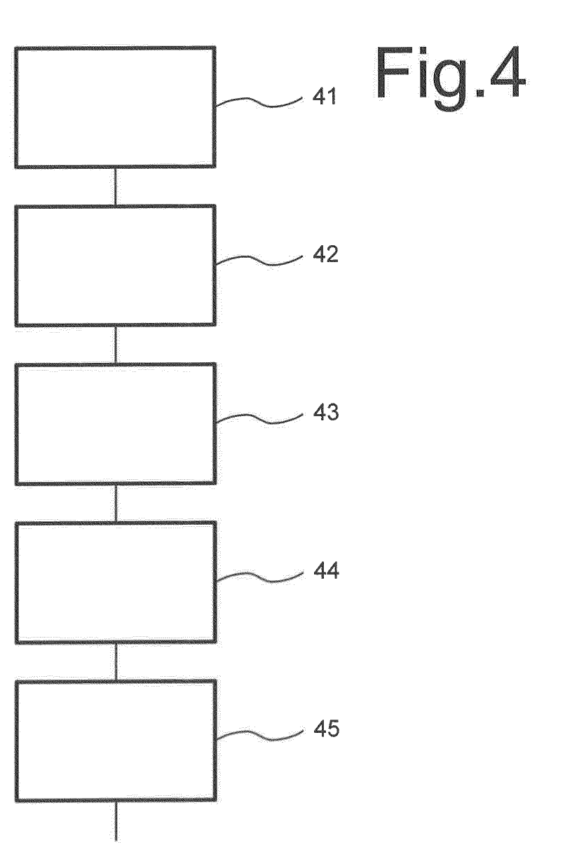

FIG. 4 shows a flow chart of an exemplary embodiment of a training process. In a first step 41 data processing system 18 determines heartbeat and respiration feature values as well as context information for each of a plurality of time intervals. Time intervals of say between thirty seconds and ten minutes distributed over an extended period of time, say between one hour and a day may be used.

The determination of the heartbeat and respiration feature values in first step 41 may be performed as described for first step 21 of FIG. 2. Optionally, feature values from other sensors, such as one or more force sensors for measuring forces on the bed may be used. In an exemplary embodiment, the context information determined in first step 41 by data processing system 18 may be based on audio data received from microphone 14, video data from camera 10 and/or force data from force sensor 16. In one example, data processing system 18 may be configured to receive audio data from microphone 14, computing an average audio power level during at least part of the time interval as a feature value (optionally the power level in a predetermined frequency band that includes frequencies produced by crying babies). In addition or alternatively, processing system 18 may determine the context information by detecting motion from images from camera 10 and determining an amplitude of the motion as a feature value (e.g. the maximum image distance between different positions of a same part of the baby's body). In addition or alternatively, processing system 18 may determine context data from signals from other sensors such as force sensor 16 by detecting maximum peak to peak force variations as a feature value.

In a second step 42, data processing system 18 determines whether for each of the time intervals whether the feature values derived from these sensors are within predefined ranges associated with an “awake” state of the baby. Optionally, data processing system 18 distinguishes between the awake state, a sleeping state and an “indeterminate” state, based on the size of the features.

In one example, data processing system 18 may be configured to compare an average or maximum audio power level feature in a time interval with a predetermined threshold, and detect that the feature value is in the predefined range if it exceeds the threshold. In addition or alternatively, processing system 18 may compare the motion amplitude feature value with a further predetermined threshold, and detect that the feature value is in the predefined range if it exceeds the further threshold. In addition or alternatively, processing system 18 may comparing the peak to peak force variation feature to a predetermined threshold, and detect that the feature value is in the predefined range if it exceeds the threshold.

In a third step 43, data processing system 18 selects a first and second set of vectors of heartbeat and respiration feature values and optional other feature values. The first set of vectors contains vectors of feature values from time intervals for which the “awake state” was determined in second step 42. The second set of feature values contains vectors of feature values from time intervals for which this was not so.

In a fourth step 44 of this exemplary embodiment, data processing system 18 execute clustering process to form clusters of feature vectors of heartbeat and respiration feature values and optional other feature values from the selected first and second set. In an embodiment, data processing system 18 first executes a clustering process for the first set, the resulting clusters of which will be referred to as “awake” clusters. Next data processing system 18 tests vectors from the second set to determine whether they lie within the “awake” clusters that were formed based on the first set (or whether they lie at no more than a predetermined distance from the centers of these “awake” clusters). If so, data processing system 18 removes the vector from the second set. In this embodiment, data processing system 18 subsequently executes a clustering process for the remaining vectors in the second set. This results in a second type of clusters, which will be referred to as “sleep” clusters. Instead of such a two step clustering, a one step clustering process may be used that requires that part of the clusters contain substantially no clusters from the second set formed in third step 43. Clusters from this part are then referred to as “sleep” clusters. Optionally, data processing system 18 may create initial clusters (seeds) for one or more of the sleeping states already by means of feature vectors from time intervals that were assigned to a definite sleeping state in second step 42. In this way, feature vectors from time intervals that found to be indeterminate in second step 42 can be added a definite sleeping state based on heartbeat and respiration feature values and optional other feature values.

Clustering methods are known per se. Clustering makes use of a distance measure between feature values or vectors of values for different features in different training instances. In an exemplary form of clustering each cluster contains feature vectors that are less distant from a reference feature vector for the cluster than to the reference feature vectors for the other clusters. An embodiment of clustering methods select reference feature vectors that minimize the combination of distances from the feature vectors of training examples to the reference feature vector of their cluster. For one dimensional feature vectors, clustering may merely be a matter of selecting reference values corresponding to peaks in the distribution of the vector values. In the present case, a feature vector comprises values of heartbeat and respiration features and optional other feature the same time interval, and the distance between such feature vector from different time intervals is used.

In an embodiment, data processing system 18 may be configured to use the clusters for current or previous assignment as initial clusters in the clustering process, e.g. to select adapted versions of the clusters iteratively so as to reduce the distance between the cluster and the training example.

In one embodiment a Euclidean distance may be used, i.e. the square root of an optionally weighted sum of squares of differences between the values of corresponding features from different time intervals. In these or other embodiments, difference measures between histograms used as features for different time intervals may be used instead of differences. Other types of difference measures, such as (weighted) sums of absolute values may also be used.

In a fifth step 45, data processing system 18 assigns the “sleep” clusters to sets of quiet sleep and active sleep clusters. This may be done for example based on assigning clusters with vectors that have an average heart rate above and below a threshold to the set of quiet sleep clusters and the set of active sleep clusters respectively. In this embodiment data processing system 18 may use the reference feature vectors for the clusters in the second step 22 of the process of FIG. 2 to assigns sleep states. Second step 22 may comprise computing distances between the feature vector determined from the measurement time interval and the reference feature vectors of the clusters and using the sleep state of the cluster at the lowest distance, or that of a cluster for which the distance is below a threshold. To detect an indication of reduced reliability of such an assignment data processing system 18 may test the distance between the reference feature vector for a cluster and an average for a plurality of time intervals of the feature vectors assigned to the corresponding sleep state according to that cluster. If this difference exceeds a predetermined threshold, data processing system 18 may trigger retraining.

As noted the embodiment described by reference to FIG. 4 is merely one example of a training process. Any type of training process, e.g. not necessarily clustering processes, for distinguishing classes in a set of training vectors and determining parameters to identify the class on that identifies trains criteria for assigning vectors to the classes may be used. The classes may be associated afterwards with different sleep states and the awake state, e.g. by determining which of the classes contain mostly the feature vectors associated with awake states, and using average heart rate and/or respiration rate to distinguish quiet sleep classes from active sleep classes. In other embodiments partially supervised training processes may be used, wherein an indication of the class is needed for only part of the classes are needed.

In a further embodiment, detection that the baby was not in a sleep state can be used to assign definite states in a time sequence model such as the hidden Markov model of FIG. 3. Subsequently, the model can be used to assign subsequent states with higher reliability than without the detection. Even if the parameters of the model need re-training because they are becoming outdated due to development of the baby this may be used to produce state assignments or probabilities for exemplary feature value measurements for use in supervised training for a limited time period after the detection.

In a simple example, detection that the baby has been put in bed, or has stopped crying, or has stopped making large movements can be used to obtain predetermined probabilities for the different possible states in the immediately subsequent time interval, given this detection. The probability that the baby is in the active sleep state given such a detection is substantially higher than that probability at an arbitrary time. This may be used to improve the reliability of supervised training with exemplary feature values obtained from that subsequent time interval. In a simple embodiment the state associated with the exemplary feature values during a predetermined time interval may be set to the active sleep state for a time interval of a predetermined duration (e.g. between one and ten minutes) for the purpose of training. Although there is a low probability that this may give rise to erroneous examples, training processes are robust for such erroneous examples.

It should be emphasized that this embodiment is merely one example of a training process that makes use of temporal relations to detected context information. By way of example, flow chart of a training process will be given for this example.

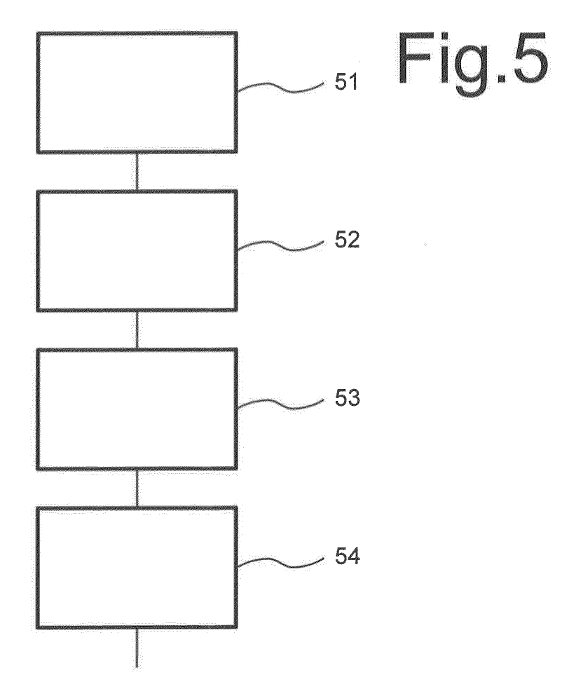

FIG. 5 shows a flow chart of an exemplary embodiment of a training process that uses such temporal information. In a first step 51, similar to first step 41 of FIG. 4, data processing system 18 determines heartbeat and respiration feature values as well as context information for each of a plurality of time intervals.

In a second step 52, data processing system 18 determines whether for each of the time intervals whether the feature values derived from these sensors are within predefined ranges associated with an “awake” state of the baby, or putting the baby in bed. These time intervals will be called seed intervals.

In a third step 53, data processing system 18 uses the detected time intervals of second step 52 to assign states to part of the other time intervals. In another embodiment, state probabilities may be assigned to these other time intervals. In general, time intervals that follow within a predetermined delay after a seed interval may be assigned to an “active sleep” state.

In a fourth step 54, data processing system 18 executes clustering process to form clusters of feature vectors of heartbeat and respiration feature values and optional other feature values from the selected first and second set. In an embodiment, data processing system 18 may first execute clustering processes for time intervals to which the “awake” state and the “active sleep states” have been assigned. Next data processing system 18 tests feature vectors from remaining time intervals to determine whether they lie within the “awake” clusters or “active” clusters (or whether they lie at no more than a predetermined distance from the centers of these clusters). Data processing system 18 subsequently executes a clustering process for the remaining vectors that are none of these clusters. The final resulting clusters are then associated with the “active sleep” states. When no movement is detected after a certain time interval after the onset of the active sleep state, the quiet sleep state is assigned.

As noted, this embodiment is merely one example of a training process that makes use of temporal relations to detected context information. As for the process of FIG. 4 any type of training process, e.g. not necessarily clustering processes, may be used for distinguishing classes in a set of training vectors and determining parameters to identify the class on that identifies trains criteria for assigning vectors to the classes. The classes may be associated afterwards with different sleep states. In other embodiments partially supervised training processes may be used, wherein an indication of the class is needed for only part of the classes are needed.

If state probabilities are used, a first set of predetermined probabilities for different states may defined for time intervals immediately following see intervals and as well as a second set of background probabilities and functions that describe how the probabilities change from the first set to the second set as a function of time distance after the seed intervals. Such sets and functions may be computed from the parameters of the Markov model. Data processing system 18 may assign the probabilities to time intervals after the seed intervals according to these functions. In such an embodiment a training processes with supervision in terms of probabilities of states may be used.

If a hidden Markov model is used, the transition probabilities of the state transitions according to this model may be re-trained based on states assigned based on the trained classifications.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A sleep monitor for monitoring baby sleep, the sleep monitor comprising
a heartbeat feature detector and/or a respiration feature detector;
a heartbeat feature and/or respiration feature based sleep state classifier with an input coupled to the heartbeat feature detector and/or the respiration feature detector;
at least one of a sound feature detector, a movement feature detector and an open eye detector;
a processing circuit configured to repeatedly execute a retraining process of the sleep state classifier during use of the sleep monitor, wherein the processing circuit is configured to detect time instants whereat the baby in the bed is in a wake state based on signals from the at least one of a sound feature detector, movement feature detector and the open eye detector, and to use the detected time instants to generate or select training examples for the retraining process.

2. A sleep monitor according to claim 1, wherein the processing circuit is configured to detect the wake state based on whether a movement amplitude of a baby motion feature detected by the movement feature detector exceeds a first predetermined value, a loudness property of sound detected by the sound feature detector exceeds a second predetermined value, and/or the open eye detector detects an open eye on the baby in the bed.

3. A sleep monitor according to claim 1, comprising a movement feature detector, the processing circuit being configured to detect the wake state based at least on whether a movement amplitude of a baby movement feature detected by the movement feature detector exceeds a first predetermined value, the sleep state classifier having an input coupled to the movement feature detector, the sleep state classifier being configured to classify sleep states based on a value or values the heartbeat feature and/or the respiration feature and a value of the baby movement feature or a further baby movement feature detected by the movement feature detector.

4. A sleep monitor according to claim 1, wherein the processing circuit is configured to perform the retraining process comprising retraining classification of a plurality of sleeping states by the sleep state classifier, the processing circuit being configured to exclude a training example for use to train classification criteria for distinguishing between said plurality of sleeping states based on whether a measurement time interval used to obtain the training example comprises at least one of the detected time instants.

5. A sleep monitor according to claim 1, wherein the sleep state classifier is configured to assign measurement time intervals to sleep states from a wake state and a first sleeping state and a second sleeping state corresponding to quiet baby sleep and active baby sleep respectively, based at least on a value or values of the heartbeat feature, and/or the respiration feature obtained for said measurement time interval, the processing circuit being configured to provide training examples associated with the first sleeping state using heartbeat feature and/or respiration feature values obtained for training time intervals that follow directly after the detected time instants whereat the baby is in a nonsleep state.

6. A method of automatically monitoring baby sleep, the method comprising
detecting heartbeat features, movement features and/or respiration features of a baby for successive measurement time intervals;
automatically classifying sleep states of the baby associated with the successive measurement time intervals based on the heartbeat and/or respiration features of the measurement time intervals;
automatically repeatedly retraining the classification criteria used for said classifying during use, said retraining comprising
detecting time instants whereat the baby in the bed is in a wake state based on signals from at least one of a sound feature detector, a movement feature detector and an open eye detector,
using the detected time instants to generate or select training examples for the retraining.

7. A method according to claim 6, wherein said detecting of the time instants comprises detecting whether a movement amplitude of a baby movement feature detected by the movement feature detector exceeds a first predetermined value, a loudness property of sound detected by the sound feature detector exceeds a second predetermined value, and/or the open eye detector detects an open eye on the baby in the bed.

8. A method according to claim 6, comprising
detecting the wake state based at least on whether a movement amplitude of a baby movement feature detected by the movement feature detector exceeds a first predetermined value,
classifying sleep states based on a value or values the heartbeat feature and/or the respiration feature and on a value of the baby movement feature or a further baby movement feature detected by the movement feature detector.

9. A method according to claim 6, wherein said retraining comprises retraining the classification criteria of a plurality of sleeping states, the method comprising excluding training examples for use to train the classification criteria for distinguishing between said plurality of sleeping states based on whether a measurement time interval used to obtain the training example comprises at least one of the detected time instants.

10. A method according to claim 6, wherein said automatically classifying sleep states comprises assigning the measurement time intervals to sleep states from a wake state and sleeping states comprising a first sleeping state and a second sleeping state corresponding to active baby sleep and quiet baby sleep respectively, said retraining comprising providing training examples associated with the first sleeping state using heartbeat feature and/or respiration feature values obtained for training time intervals that follow directly after the detected time instants whereat the baby is in a non-sleep state.

11. A computer program product, comprising instructions for a programmable data processing system that, when executed by the data processing system, will cause the data processing system to execute the method of claim 6.
